(19) United States
(12) Patent Application Publication
(10) Pub. No.: US 2022/0306713 A1
(43) Pub. Date: Sep. 29, 2022

(54) ANTIBODY-INTERLEUKIN FUSION PROTEIN AND METHODS OF USE

(71) Applicant: APOLLOMICS INC. (HANGZHOU), Hangzhou, Zhejiang (CN)

(72) Inventors: Lan YANG, Hangzhou, Zhejiang (CN); Guoliang YU, Incline Village, NV (US); Qian SHI, Hangzhou, Zhejiang (CN); Zhongwei FEI, Hangzhou, Zhejiang (CN); Biao MA, Hangzhou, Zhejiang (CN)

(21) Appl. No.: 17/617,945

(22) PCT Filed: Jun. 10, 2020
(86) PCT No.: PCT/CN2020/095354

§ 371 Date: Dec. 10, 2021

(30) Foreign Application Priority Data
Jun. 10, 2019 (CN) PCT/CN2019/090494

(51) Int. Cl. C07K 14/54; C07K 16/28; A61P 35/00

(52) U.S. Cl. CPC C07K 14/5428; C07K 16/2818; C07K 16/2827; A61P 35/00; C07K 2319/30; A61K 2039/505

(57) ABSTRACT

Provided is a protein comprising an antibody or antigen-binding fragment and an interleukin molecule operably linked to the antibody or antigen-binding fragment. The antibody or antigen-binding fragment specifically binds to an immune checkpoint protein. The interleukin molecule is IL-10.

### CROSS-REFERENCE TO RELATED APPLICATIONS

This application claims priority to PCT/CN2019/090494, filed Jun. 10, 2019, the disclosure of which is incorporated herein by reference.

### FIELD OF THE INVENTION

The present disclosure relates generally to the fields of biology, therapeutics and diagnostics. In particular, the disclosure relates to antibody-interleukin fusion proteins and uses thereof.

### BACKGROUND

Cancer treatment has made significant progress in recent years as understanding of the underlying biological processes has increased. For example, the development of immunotherapies that induce the patient's own immune system to fight the tumor highlights the significance of the mechanisms that promote immune tolerance to tumor antigens expressed by cancer-associated genetic alteration. These immune checkpoint inhibitors, represented by monoclonal antibodies against PD-1, PD-L1 or CTLA4, have yielded remarkable and durable responses for some patients with an increasingly broad array of cancer types. However, current immunotherapies, such as PD-1 or PD-L1 blockade, only exhibit limited response in cancer patients (see, e.g., P Sharma and JP Allison, Cell (2015) 161: 205-214), and chemotherapy resistance is still one of the most pressing major dilemmas in cancer therapy. Therefore, there is a continuing need to develop new compositions and methods to modulate immune system and eliminate tumor immunosuppressive effects in order to address tumor immune tolerance and chemotherapy resistance.

### SUMMARY

In one aspect, the present disclosure provides a fusion protein. In one embodiment, the protein comprises: an antibody or antigen-binding fragment comprising a heavy chain variable domain, and a light chain variable domain; and an interleukin molecule operably linked to the antibody or antigen-binding fragment.

In certain embodiments, the antibody or antigen-binding fragment specifically binds to an immune checkpoint protein. In certain embodiments, the immune checkpoint protein is selected from the group consisting of A2AR, B7.1, B7.2, B7-H2, B7-H3, B7-H4, B7-H6, BTLA, CD48, CD120b, CD160, CD244, CTLA-4, ICOS, LAG-3, LILRB1, LILRB2, LILRB4, OX40, PD-1, PD-L1, PD-L2, S1RPalpha (CD47), TIGIT, TIM-3, TIM-1, TIM-4, and VISTA. In certain embodiments, the immune checkpoint is PD-1 or PD-L1.

In certain embodiments, the antibody comprises a Fc region. In certain embodiments, the antigen-binding fragment is an F(ab′)2 fragment. In certain embodiments, the antigen-binding fragment is a single chain variable fragment (scFv).

In certain embodiments, the interleukin molecule is selected from the group consisting of IL-1, IL-2, IL-3, IL-4, IL-5, IL-6, IL-7, IL-8, IL-IL-9, IL-10, IL-11, IL-12, IL-13, IL-14, IL-15, IL-16, IL-17, IL-18, IL-19, IL20, IL-21, IL-22, IL-23, IL-24, IL-25, IL-26, IL-27, IL-28, IL-29, IL-30, IL-31, IL-32, IL-33, IL-35 and IL-36. In certain embodiments, the interleukin molecule is IL-10.

In certain embodiments, the interleukin molecule is linked to the heavy chain variable domain. In certain embodiments, the interleukin molecule is linked to N-terminal of the heavy chain variable domain. In certain embodiments, the interleukin molecule is linked to C-terminal of the heavy chain variable domain. In certain embodiments, the interleukin molecule is linked to the light chain variable domain. In certain embodiments, the interleukin molecule is linked to N-terminal of the light chain variable domain. In certain embodiments, the interleukin molecule is linked to C-terminal of the light chain variable domain.

In certain embodiment, the protein described further comprises a linker that links the antibody or antigen-binding fragment thereof and the interleukin molecule. In certain embodiments, the linker comprising the amino acid sequence (GGGGS)n (n=2-5).

In another aspect, the present disclosure provides an isolated polynucleotide encoding the protein described herein.

In another aspect, the present disclosure provides a vector capable of expressing a protein, comprising the isolated polynucleotide described herein.

In another aspect, the present disclosure provides a recombinant host cell suitable for producing a protein, comprising the vector described herein. In certain embodiments, the recombinant host cell is a mammalian cell line. In certain embodiments, the mammalian cell line is a CHO cell line.

In another aspect, the present disclosure provides a pharmaceutical composition comprising the protein described herein and a pharmaceutically acceptable carrier.

In another aspect, the present disclosure provides a method for treating a disease in a subject in need thereof, comprising administering to the subject a therapeutically amount of the pharmaceutical composition described herein. In certain embodiments, the disease is a tumor. In certain embodiments, the tumor is selected from the groups consisting of a lung cancer, a melanoma, a renal cancer, a liver cancer, a myeloma, a prostate cancer, a breast cancer, a colorectal cancer, a pancreatic cancer, a thyroid cancer, a hematological cancer, a leukemia and a non-Hodgkin's lymphoma.

In certain embodiments, the disease is an immune related disorder. In certain embodiments, the immune related disorder is selected from the group consisting of inflammatory bowel disease, Crohn's disease, ulcerative colitis, rheumatoid arthritis, psoriasis, type I diabetes, acute pancreatitis, uveitis, Sjogren's disease, Behcet's disease, sarcoidosis, graft versus host disease (GVHD), System Lupus Erythematosus, Vitiligo, chronic prophylactic acute graft versus host disease (pGvHD), HIV-induced vasculitis, Alopecia areata, Systemic sclerosis morphoea, and primary anti-phospholipid syndrome.

The use of the word “a” or “an” when used in conjunction with the term “comprising” in the claims and/or the specification may mean “one,” but it is also consistent with the meaning of “one or more,” “at least one,” and “one or more than one.”

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description.

### BRIEF DESCRIPTION OF DRAWING

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrate an exemplary embodiment of the fusion protein of the invention.

FIG. 2 illustrate an exemplary embodiment of the fusion protein of the invention.

FIG. 3 illustrate an exemplary embodiment of the fusion protein of the invention.

FIG. 4 illustrate an exemplary embodiment of the fusion protein of the invention.

FIG. 5 illustrate an exemplary embodiment of the fusion protein of the invention.

FIG. 6 illustrate an exemplary embodiment of the fusion protein of the invention.

FIG. 7 illustrate an exemplary embodiment of the fusion protein of the invention.

FIG. 8 illustrate an exemplary embodiment of the fusion protein of the invention.

FIGS. 9A and 9B illustrate the induction of cytokine production in a Mixed Lymphocyte Reaction (MLR) elicited by anti-PD-1 (7A4D), anti-PD-L1 (5G11) monoclonal antibody and the derived antibody-IL-10 fusion proteins. LC, IL-10 linked at the Ig light chain C-terminus; HC, IL-10 linked at the Ig heavy chain C-terminus.

FIG. 10 illustrates the MC/9 cell proliferation elicited by antibody-IL-10 fusion proteins based on anti-PD-1 (7A4D) and anti-PD-L1(5G11) monoclonal antibody.

FIG. 11 illustrates the characterization of binding of anti-PD-L1 (10F.9G2) antibody and anti-PD-L1 (10F.9G2) antibody-HC-mIL-10 fusion protein to PD-L1-expressing mouse tumor cells. Mouse tumor cell line A20 and CT26 express PD-L1 on the surface at a very high and low level, respectively. Anti-PD-L1 (10F.9G2) antibody and anti-PD-L1(10F.9G2) antibody-HC-mIL-10 fusion protein were each labelled with PE using a R-Phycoerythrin Conjugation Kit (Abcam). Prior to staining, A20 and CT26 cells were treated with 1 μg/mL Mouse BD Fc Block (BD Biosciences) at 4° C. for 5 min. Various amount of PE-labelled proteins as specified was used to stain treated cells in a total volume of 100 μl. Stained cells were analyzed on a LSRFortessa X-20 Flow Cytometer. The left and right panels show the data from CT26 and A20 cells, respectively.

FIG. 12 illustrates the effects of anti-PD-L1 antibody-HC-mIL-10 fusion protein on syngeneic CT26 mouse tumor model.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### I. Definition

It is to be understood that both the foregoing general description and the following detailed description are exemplary and explanatory only and are not restrictive of the invention as claimed. In this application, the use of the singular includes the plural unless specifically stated otherwise. In this disclosure, the term “or” is used to mean “and/or” unless explicitly indicated to refer to alternatives only or the alternatives are mutually exclusive. As used herein “another” may mean at least a second or more. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

As used herein, the term “administering” means providing a pharmaceutical agent or composition to a subject, and includes, but is not limited to, administering by a medical professional and self-administering.

The term “antibody” as used herein includes any immunoglobulin, monoclonal antibody, polyclonal antibody, multivalent antibody, bivalent antibody, monovalent antibody, multispecific antibody, bispecific antibody as well as the antigen-binding fragment thereof that binds to a specific antigen. A native intact antibody comprises two heavy (H) chains and two light (L) chains. Mammalian heavy chains are classified as alpha, delta, epsilon, gamma, and mu, each heavy chain consists of a variable domain (V_{H}) and a constant region including a first, second, and third constant domain (C_{H}1, C_{H}2, C_{H}3, respectively); mammalian light chains are classified as λ or κ, while each light chain consists of a variable domain (V_{L}) and a constant domain (C_{L}). The antibody has a “Y” shape, with the stem of the Y consisting of the second and third constant domains of two heavy chains bound together via disulfide bonding. Each arm of the Y includes the variable domain and first constant domain of a single heavy chain bound to the variable and constant domains of a single light chain. The variable domains of the light and heavy chains are responsible for antigen binding. Each variable domain of both heavy and light chains generally contains three highly variable loops called the complementarity determining regions (CDRs) (light chain CDRs including LCDR1, LCDR2, and LCDR3, heavy chain CDRs including HCDR1, HCDR2, HCDR3). CDR boundaries for the antibodies disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989); Kabat E. A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). The three CDRs of the heavy or light chain variable domain are interposed between flanking stretches known as framework regions (FRs), which are more highly conserved than the CDRs and form a scaffold to support the hypervariable loops. The constant domains of the heavy and light chains are not involved in antigen-binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

The term “antigen” refers to a substance capable of inducing adaptive immune responses. Specifically, an antigen is a substance specifically bound by antibodies or T lymphocyte antigen receptors. Antigens are usually proteins and polysaccharides, less frequently also lipids. Suitable antigens include without limitation parts of bacteria (coats, capsules, cell walls, flagella, fimbrai, and toxins), viruses, and other microorganisms. Antigens also include tumor antigens, e.g., antigens generated by mutations in tumors. As used herein, antigens also include immunogens and haptens.

The term “antigen binding fragment” as used herein refers to a portion of a protein which is capable of binding specifically to an antigen. In certain embodiment, the antigen binding fragment is derived from an antibody comprising one or more CDRs, or any other antibody fragment that binds to an antigen but does not comprise an intact native antibody structure. Examples of antigen binding fragments include, without limitation, a diabody, a Fab, a Fab′, a F(ab′)₂, a Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a scFv dimer (bivalent diabody), a multispecific antibody, a single domain antibody (sdAb), a camelid antibody or a nanobody, a domain antibody, and a bivalent domain antibody. In certain embodiments, an antigen binding fragment is capable of binding to the same antigen to which the parent antibody binds.

A “Fab fragment” comprises one light chain and the C_{H}1 and variable domain of one heavy chain. The heavy chain of a Fab molecule cannot form a disulfide bond with another heavy chain molecule.

A “Fab′ fragment” comprises one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

An “Fc” region comprises two heavy chain fragments comprising the C_{H}2 and C_{H}3 domains of an antibody. The two heavy chain fragments are held together by two or more disulfide bonds and by hydrophobic interactions of the CH3 domains. The Fc region of the antibody is responsible for various effector functions such as antibody-dependent cell-mediated cytotoxicity (ADCC), and complement dependent cytotoxicity (CDC), but does not function in antigen binding.

The “Fv region” comprises the variable domains from both the heavy and light chains but lacks the constant domains.

“Single-chain antibodies” or “single-chain Fvs” or “scFv” are Fv molecules in which the heavy and light chain variable domains have been connected by a flexible linker to form a single polypeptide chain, which forms an antigen binding region. Single chain antibodies are discussed in detail in International Patent Application Publication No. WO 88/01649 and U.S. Pat. Nos. 4,946,778 and 5,260,203, the disclosures of which are incorporated by reference.

“Single-chain Fv-Fc antibody” or “scFv-Fc” refers to an engineered antibody consisting of a scFv connected to the Fc region of an antibody.

A “dsFv” refers to a disulfide-stabilized Fv fragment that the linkage between the variable domain of a single light chain and the variable domain of a single heavy chain is a disulfide bond. In some embodiments, a “(dsFv)₂” or “(dsFv-dsFv′)” comprises three peptide chains: two V_{H} domains linked by a peptide linker (e.g., a long flexible linker) and bound to two V_{L} domains, respectively, via disulfide bridges. In some embodiments, dsFv-dsFv′ is bispecific in which each disulfide paired heavy and light chain has a different antigen specificity.

“Camelized single domain antibody,” “heavy chain antibody,” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (“VHH domain”) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of one V_{H} domain from a heavy chain antibody of a conventional IgG, and two heavy chain constant domains, for example C_{H}2 and C_{H}3.

“Diabodies” or “dAbs” include small antibody fragments with two antigen-binding sites, wherein the fragments comprise a V_{H} domain connected to a V_{L} domain in the same polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

In certain embodiments, a “scFv dimer” is a bivalent diabody or bivalent ScFv (B sFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or epitopes) or different antigens (or epitopes). In other embodiments, a “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

A “domain antibody” is an immunologically functional immunoglobulin fragment containing only the variable domain of a heavy chain or the variable domain of a light chain. In some instances, two or more V_{H} domains are covalently joined with a peptide linker to create a bivalent domain antibody. The two V_{H} domains of a bivalent domain antibody can target the same or different antigens.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal such as mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions are derived from human.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

A protein or antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope. For example, the antibody described herein can specifically bind to an immune checkpoint protein. In some embodiments, the antibody that binds to the immune checkpoint protein has a dissociation constant (K_{d}) of ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

As used herein, the term “cancer” and “tumor” are used interchangeably and refer to any diseases involving an abnormal cell growth and includes all stages and all forms of the disease that affects any tissue, organ or cell in the body. The term includes all known tumors and neoplastic conditions, whether characterized as malignant, benign, soft tissue, or solid, and cancers of all stages and grades including pre- and post-metastatic cancers. In general, tumors can be categorized according to the tissue or organ from which the tumor is located or originated and morphology of cancerous tissues and cells. As used herein, tumor types include, acute lymphoblastic leukemia (ALL), acute myeloid leukemia, adrenocortical carcinoma, anal cancer, astrocytoma, childhood cerebellar or cerebral, basal-cell carcinoma, bile duct cancer, bladder cancer, bone tumor, brain cancer, breast cancer, Burkitt's lymphoma, cerebellar astrocytoma, cerebral astrocytoma/malignant glioma, cervical cancer, chronic lymphocytic leukemia, chronic myelogenous leukemia, colon cancer, emphysema, endometrial cancer, ependymoma, esophageal cancer, Ewing family of tumors, Ewing's sarcoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, leukaemia, liver cancer, lung cancer, medulloblastoma, melanoma, neuroblastoma, non-Hodgkin lymphoma, ovarian cancer, pancreatic cancer, pharyngeal cancer, prostate cancer, rectal cancer, renal cell carcinoma (kidney cancer), retinoblastoma, skin cancer, stomach cancer, supratentorial primitive neuroectodermal tumors, testicular cancer, throat cancer, thyroid cancer, vaginal cancer, visual pathway and hypothalamic glioma.

As used herein, the term “effective amount” or “therapeutically effective amount” means the amount of agent that is sufficient to prevent, treat, reduce and/or ameliorate the symptoms and/or underlying causes of any disorder or disease, or the amount of an agent sufficient to produce a desired effect on a cell. In one embodiment, a “therapeutically effective amount” is an amount sufficient to reduce or eliminate a symptom of a disease. In another embodiment, a therapeutically effective amount is an amount sufficient to overcome the disease itself.

The term “epitope” as used herein refers to the specific group of atoms or amino acids on an antigen to which an antigen binding polypeptide binds. The epitope can be either linear epitope or a conformational epitope. A linear epitope is formed by a continuous sequence of amino acids from the antigen and interacts with an antibody based on their primary structure. A conformational epitope, on the other hand, is composed of discontinuous sections of the antigen's amino acid sequence and interacts with the antibody based on the 3D structure of the antigen. In general, an epitope is approximately five or six amino acid in length. Two antibodies may bind the same epitope within an antigen if they exhibit competitive binding for the antigen.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

As used herein, an “isolated” biological component (such as a nucleic acid, peptide or cell) has been substantially separated, produced apart from, or purified away from other biological components or cells of the organism in which the component naturally occurs, i.e., other chromosomal and extrachromosomal DNA and RNA, cells and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

The term “link” as used herein refers to the association via intramolecular interaction, e.g., covalent bonds, metallic bonds, and/or ionic bonding, or inter-molecular interaction, e.g., hydrogen bond or noncovalent bonds.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The terms “polypeptide” or “protein” means a macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogs of a corresponding naturally-occurring amino acid and polymers. The terms “polypeptide” and “protein” specifically encompass LAIR1 antigen binding proteins, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. In certain embodiments, fragments are about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains. In the case of an antibody, useful fragments include but are not limited to a CDR region, a variable domain of a heavy and/or light chain, a portion of an antibody chain or just its variable region including two CDRs, and the like.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “subject” refers to a human or any non-human animal (e.g., mouse, rat, rabbit, dog, cat, cattle, swine, sheep, horse or primate). A human includes pre and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

As used herein, a “vector” refers to a nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### II. Fusion Protein and Production Thereof

A. Fusion Protein

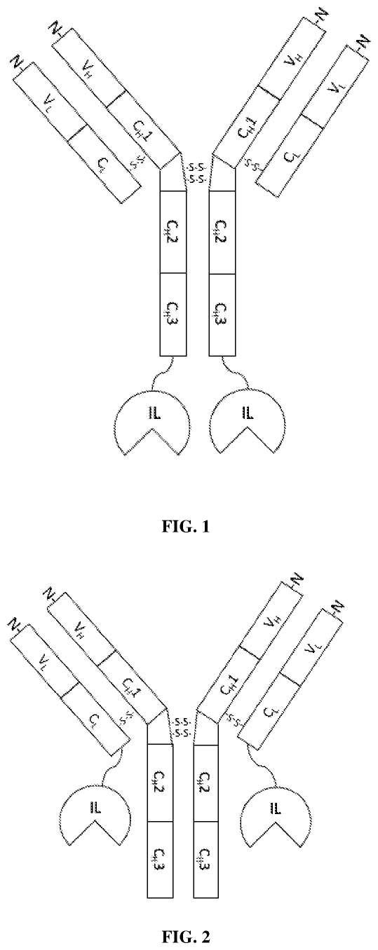

The present disclosure in one aspect provides a fusion protein that comprises: an antibody or antigen-binding fragment comprising a heavy chain variable domain, and a light chain variable domain; and an interleukin molecule operably linked to the antibody or antigen-binding fragment. It is appreciated that the fusion protein of the present disclosure can have various forms and structures. The fusion protein of the present disclosure can be understood by the exemplary embodiments as illustrated in FIGS. 1-8.

Referring to FIG. 1, in an exemplary embodiment of the invention, the fusion protein comprises a conventional Y shaped antibody that comprises two pairs of heavy chain and light chain. Each heavy chain consists of a variable domain (V_{H}) and a constant region including a first, second, and third constant domain (C_{H}1, C_{H}2, C_{H}3, respectively). Each light chain consists of a variable domain (V_{L}) and a constant domain (C_{L}). The stem of the Y shaped antibody consisting of the second and third constant domains of two heavy chains bound together via disulfide bonding. Each arm of the Y shaped antibody includes the variable domain and first constant domain of a single heavy chain bound to the variable and constant domains of a single light chain. At the C-terminal of each heavy chain, an interleukin (IL) molecule is linked to the third constant domain of the heavy chain via a linker.

Referring to FIG. 2, in another exemplary embodiment of the invention, the fusion protein comprises a conventional Y shaped antibody having the same structure as illustrated in FIG. 1. Unlike in FIG. 1, instead of linking to the heavy chain, in the fusion protein illustrated in FIG. 2, an IL molecule is linked to the constant domain of the light chain via a linker.

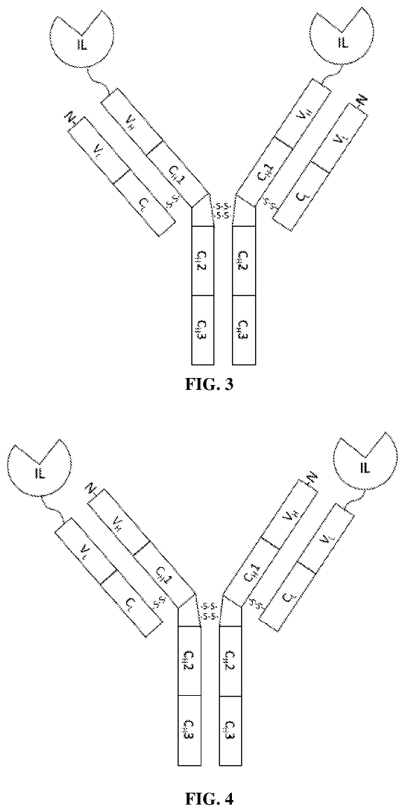

Referring to FIG. 3, in another exemplary embodiment of the invention, the fusion protein comprises a conventional Y shaped antibody having the same structure as illustrated in FIG. 1. In the fusion protein illustrated in FIG. 3, an IL molecule is linked to the heavy chain variable domain via a linker.

Referring to FIG. 4, in another exemplary embodiment of the invention, the fusion protein comprises a conventional Y shaped antibody having the same structure as illustrated in FIG. 1. In the fusion protein illustrated in FIG. 4, an IL molecule is linked to the light chain variable domain via a linker.

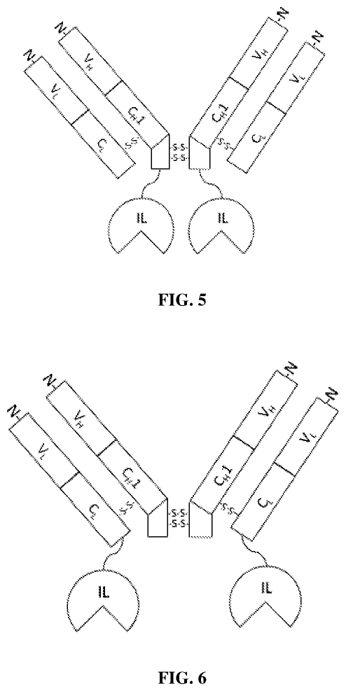

In certain embodiments, as illustrated in FIGS. 5-8, the fusion protein comprises an antigen-binding fragment. Referring to FIG. 5, in another exemplary embodiment of the invention, the fusion protein comprises an antigen-binding fragment F(ab′)₂ consisting of two pairs of light chains and heavy chain fragments. Each heavy chain fragment contains a portion of the constant region between the C_{H}1 and C_{H}2 domains, such that an interchain disulfide bond is formed between the two heavy chain fragments. At the C-terminal of each heavy chain fragment, an IL molecule is linked to the heavy chain fragment via a linker.

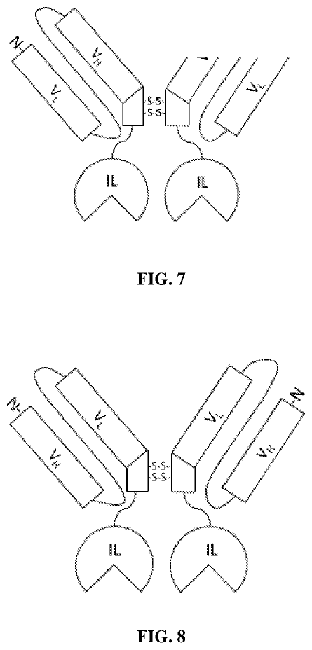

Referring to FIG. 6, in another exemplary embodiment of the invention, the fusion protein comprises a F(ab′)₂ fragment having the same structure as illustrated in FIG. 5. Unlike in FIG. 5, instead of linking to the heavy chain fragment, in the fusion protein illustrated in FIG. 6, an IL molecule is linked to the constant domain of the light chain via a linker.

Referring to FIG. 7, in another exemplary embodiment of the invention, the fusion protein comprises a pair of single chain variable fragments (scFv). scFv is a molecule in which the heavy and light chain variable domains have been connected by a flexible linker to form a single polypeptide chain, which forms an antigen binding region. Single chain variable fragments are discussed in detail in International Patent Application Publication No. WO 88/01649 and U.S. Pat. Nos. 4,946,778 and 5,260,203, the disclosures of which are incorporated by reference. Referring to FIG. 7, the heavy chain variable domain links to a portion of the heavy chain constant region between the C_{H}1 and C_{H}2 domains, such that an interchain disulfide bond is formed between the two single chain variable fragments. At the C-terminal of each scFv, an IL molecule is linked to the scFv via a linker.

Referring to FIG. 8, in another exemplary embodiment of the invention, the fusion protein comprises a pair of scFv, each scFv contains from a heavy chain variable domain and a light chain variable domain from the N-terminal to C-terminal of the polypeptide. The light chain variable domain links to a portion of the heavy chain constant region between the C_{H}1 and C_{H}2 domains, such that an interchain disulfide bond is formed between the two single chain variable fragments. At the C-terminal of each scFv, an IL molecule is linked to the scFv via a linker.

In certain embodiments, the antibody or antigen-binding fragment specifically binds to an immune checkpoint protein. In certain embodiments, the immune checkpoint protein is selected from the group consisting of A2AR, B7.1, B7.2, B7-H2, B7-H3, B7-H4, B7-H6, BTLA, CD48, CD120b, CD160, CD244, CTLA-4, ICOS, LAG-3, LILRB1, LILRB2, LILRB4, OX40, PD-1, PD-L1, PD-L2, S1RPalpha (CD47), TIGIT, TIM-3, TIM-1, TIM-4, and VISTA.

In certain embodiments, the fusion protein comprises anti-PD-1 antibody selected from those disclosed in U.S. Pat. No. 10,428,146 to Qui et al, the disclosure of which is incorporated in its entirety by reference. In certain embodiments, the anti-PD-1 antibody is 7A4D disclosed in U.S. Pat. No. 10,428,146. In certain embodiments, the fusion protein comprises an antigen-binding fragment derived from the anti-PD-1 antibodies disclosed in U.S. Pat. No. 10,428,146. In certain embodiments, the anti-PD-1 antibody or the antigen-binding fragment comprises (i) a heavy variable region having HCDR1 with an amino acid sequence of SEQ ID NO: 1, HCDR2 with an amino acid sequence of SEQ ID NO: 2, HCDR3 with an amino acid sequence of SEQ ID NO: 3; and (ii) a light variable region having LCDR1 with an amino acid sequence of SEQ ID NO: 4, LCDR2 with an amino acid sequence of SEQ ID NO: 5, LCDR3 with an amino acid sequence of SEQ ID NO: 6.

In certain embodiments, the fusion protein comprises an anti-PD-L1 antibody selected from those disclosed in U.S. Pat. No. 10,435,470 to Zha et al, the disclosure of which is incorporated in its entirety by reference. In certain embodiments, the anti-PD-L1 antibody is 5G11 disclosed in U.S. Pat. No. 10,435,470. In certain embodiments, the fusion protein comprises an antigen-binding fragment derived from the anti-PD-L1 antibodies disclosed in U.S. Pat. No. 10,435,470. In certain embodiments, the anti-PD-L1 antibody or the antigen-binding fragment comprises (i) a heavy variable region having HCDR1 with an amino acid sequence of SEQ ID NO: 7, HCDR2 with an amino acid sequence of SEQ ID NO: 8, HCDR3 with an amino acid sequence of SEQ ID NO: 9; and (ii) a light variable region having LCDR1 with an amino acid sequence of SEQ ID NO: 10, LCDR2 with an amino acid sequence of SEQ ID NO: 11, LCDR3 with an amino acid sequence of SEQ ID NO: 12.

In certain embodiments, the interleukin molecule is selected from the group consisting of IL-1, IL-2, IL-3, IL-4, IL-5, IL-6, IL-7, IL-8, IL-IL-9, IL-10, IL-11, IL-12, IL-13, IL-14, IL-15, IL-16, IL-17, IL-18, IL-19, IL20, IL-21, IL-22, IL-23, IL-24, IL-25, IL-26, IL-27, IL-28, IL-29, IL-30, IL-31, IL-32, IL-33, IL-35 and IL-36. In certain embodiments, the interleukin molecule is IL-7, IL10 or IL-23. In certain embodiments, the interleukin molecule is a human interleukin.

Interleukin 7 (IL-7) is a hematopoietic growth factor secreted by stromal cells in the bone marrow and thymus. It is also produced by keratinocytes, dendritic cells, hepatocytes, neurons, and epithelial cells, but is not produce by normal lymphocytes. Human IL-7 mRNA has a sequence of GenBank Reference No. NM_000880, NM_001199886, NM_001199887, and NM_001199888. Human IL-7 protein has a sequence of GenBank Reference No. NP_000871, NP_001186815, NP_001186816, and NP_001186817. IL-7 stimulates the differentiation of multipotent (pluripotent) hematopoietic stem cells into lymphoid progenitor cells. It also stimulates proliferation of all cells in the lymphoid lineage, including B cells, T cells and NK cells.

Interleukin 10 (IL-10), also known as human cytokine synthesis inhibitory factor (CSIF), is generally regarded as an anti-inflammatory cytokine. Human IL-10 protein is a homodimer, each of its subunits is 178 amino acid long. Human IL-10 mRNA has a sequence of GenBank Reference No. NM_000572. Human IL-10 protein has a sequence of GenBank Reference No. NP_000563. In certain embodiments, the IL-10 molecule comprised in the fusion protein has an amino acid sequence of SEQ ID NO: 13 or a sequence having at least 80%, 85%, 90%, 91%, 92%, 93%, 94%, 95%, 96%, 97%, 98%, 99% identity thereto. In certain embodiments, the IL-10 molecule comprised in the fusion protein has an amino acid sequence of SEQ ID NO: 14 or a sequence having at least 80%, 85%, 90%, 91%, 92%, 93%, 94%, 95%, 96%, 97%, 98%, 99% identity thereto.

“Percentage of identity” in the context of polypeptide or polynucleotide is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

IL-10 signals through a receptor complex consisting of two IL-10 receptor-1 and two IL-10 receptor-2 proteins. IL-10 binding induces STAT3 signaling via the phosphorylation of the cytoplasmic tails of IL-10 receptor 1 plus IL-10 receptor 2 by JAKI and Tyk2. IL-10 is a cytokine with multiple pleiotropic effects in immunoregulation and inflammation. It downregulates the expression of Th1 cytokines, MHC class II antigen, and co-stimulatory molecules on macrophages. It also enhances B cell survival, proliferation, and antibody production.

Interleukin 23 (IL-23) is a heterodimeric cytokine composed of an IL12B subunit and the IL23A subunit. IL-23 maintains IL-17 producing cells, increases angiogenesis and reduces CD8 T-cell infiltration.

In certain embodiments, the fusion protein contains a linker that links the interleukin molecule to the antibody or antigen-binding fragment. In certain embodiment, the linkers generally are comprised of helix- and turn-promoting amino acid residues such as alanine, serine and glycine. However, other residues can function as well. In certain embodiments, the linker comprising the amino acid sequence (GGGGS)n (n=2-5).

B. Methods of Production

The fusion protein according to the present disclosure can be prepared recombinantly, by expression from e.g. a nucleic acid construct encoding for the fusion protein, for example as described in Antibody Engineering: Methods and Protocols, Second Edition (Humana Press, 2012), at Chapter 40: Production of Bispecific Antibodies: Diabodies and Tandem scFv (Hornig and Farber-Schwarz).

In certain embodiments, the fusion protein can be prepared based on monoclonal antibodies against a target antigen, e.g., an immune checkpoint protein, which can be prepared using standard methods, followed by screening, characterization and functional assessment. Variable regions of the monoclonal antibodies can be sequenced and then subcloned into an expression vector to produce the genes encoding the fusion protein, which are then expressed and purified.

The fusion protein may further be engineered by a process of affinity maturation in which a modified antibody or antigen-binding fragment is generated that has an improvement in the affinity of binding to the antigen, compared to an unmodified parent antibody or antigen-binding fragment. Affinity-matured antibody or antigen-binding fragment may be produced by procedures known in the art, e.g., Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):331 0-15 9 (1995); and Hawkins of al, J. Mol. Biol. 226:889-896 (1992).

The fusion protein that contains a conventional Y shaped antibody as disclosed herein may also be engineered to include modifications within the Fc region, typically to alter one or more functional properties of the antibody, such as serum half-life, complement fixation, Fc receptor binding, and/or effector function (e.g., antigen-dependent cellular cytotoxicity). Furthermore, the antigen binding polypeptide disclosed herein can be chemically modified (e.g., one or more chemical moieties can be attached to the antibody) or be modified to alter its glycosylation, again to alter one or more functional properties of the antigen binding polypeptide. The antibodies disclosed herein also include antigen binding polypeptides with modified (or blocked) Fc regions to provide altered effector functions. See, e.g., U.S. Pat. No. 5,624,821; WO2003/086310; WO2005/120571; WO2006/0057702. Such modification can be used to enhance or suppress various reactions of the immune system, with possible beneficial effects in diagnosis and therapy.

In certain embodiments, scFv contained in the fusion protein can be created through phage display where it is highly convenient to express the antigen binding domain as a single peptide. In general, a random linker library can be constructed in which the genes for the heavy and light chain variable domains are linked by a segment encoding an 18-amino acid polypeptide of variable composition. The scFv repertoire (approx. 5×10⁶ different members) is displayed on filamentous phage and subjected to affinity selection with the target antigen, e.g., IL-10.

Alternatively, scFv can be created directly from subcloned heavy and light chains derived from a monoclonal antibody.

In certain embodiments, the fusion protein of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins (e.g., by weight) in the composition.

### III. Compositions and Conjugates

A. Formulation

The present disclosure provides pharmaceutical compositions comprising the fusion protein described herein. Such compositions comprise a prophylactically or therapeutically effective amount of an antigen binding polypeptide, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antigen binding polypeptides, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

The fusion protein of the present disclosure, as described herein, can be formulated for parenteral administration, e.g., formulated for injection via the intradermal, intravenous, intramuscular, subcutaneous, intra-tumoral or even intraperitoneal routes. The antigen binding polypeptides could alternatively be administered by a topical route directly to the mucosa, for example by nasal drops, inhalation, or by nebulizer. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Generally, the ingredients of compositions of the present disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the present disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

B. Conjugates

The fusion protein of the present disclosure may be linked to at least one agent to form a conjugate. In order to increase the efficacy of the fusion protein as therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides.

In certain embodiments, the conjugates of the present disclosure include drug conjugates comprising the fusion protein that are covalently linked to drugs. In certain embodiments, the conjugates are used to treat diseases, for example, by administering the conjugates to a subject having a disease. Examples of suitable drugs include anti-tumor drugs, antibiotics, and immunosuppressive drugs.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the effector molecule is conjugated to a carbohydrate residue in the Fc region have also been disclosed in the literature (O'Shannessy et al., 1987).

### IV. Diseases Treated by the Fusion Protein

In one aspect, the present disclosure provides methods of using the fusion protein as disclosed herein to treat diseases, including without limitation, tumors and immune disorders.

A. Tumors

While hyperproliferative diseases can be associated with any disease which causes a cell to begin to reproduce uncontrollably, the prototypical example is tumor. One of the key elements of tumor is that the cell's normal apoptotic cycle is interrupted and thus agents that interrupt the growth of the cells are important as therapeutic agents for treating these diseases. Here, a fusion protein of interest can be generated against an antigen on the surface of the tumor cell, and in particular on the surface of cancer stem cells, or in the presence of immune cells which are inhibited by the presence of such antigen.

Tumor cells that may be treated according to the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the tumor may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

B. Immune Disorders

Immune disorders are resulted from dysfunction of immune system. Fusion proteins against an antigen that regulates immune cells, such as B cells, T cell, NK cells etc., can be used to modulate the immune cells, thereby treating immune disorders, such as inflammation, autoimmune diseases and transplant rejection.

An autoimmune disease, as used herein, refers to a condition arising from an abnormal immune response to a normal body part. There are more than 80 illnesses caused by autoimmune diseases. Nearly any body part can be involved. Autoimmune diseases have a wide variety of different effects, including damage to or destruction of tissues, altered organ growth and altered organ function. About 24 million (7%) people in the United States are affected by an autoimmune disease.

Some common diseases that are considered as an autoimmune disease include alopecia areata, ankylosing spondylitis, antiphospholipid syndrome, autoimmune Addison's disease, autoimmune hemolytic anemia, autoimmune hepatitis, autoimmune inner ear disease, autoimmune lymphoproliferative syndrome (ALPS), autoimmune thrombocytopenic purpura (ATP), Behcet's disease, bullous pemphigoid, cardiomyopathy, celiac disease, celiac sprue-dermatitis, chronic fatigue immune deficiency syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis-juvenile, discoid lupus, essential mixed cryoglobulinemia, fibromyalgia-fibromyositis, grave's disease, Guillain-Barre, Hashimoto's thyroiditis, idiopathic pulmonary fibrosis, idiopathic thrombocytopenia purpura (ITP), IgA nephropathy, inflammatory bowel disease, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatic, polymyositis and dermatomyositis, primary biliary cirrhosis, psoriasis, Raymond's phenomenon, Reiter's syndrome, rheumatic fever, rheumatoid arthritis, sarcoidosis, scleroderma, Sjogren's syndrome, stiff-man syndrome, systemic lupus erythematosus, Takayasu arteritis, temporal arteritis/giant cell arteritis, type 1 diabetes, ulcerative colitis, uveiitis, vitiligo, and Wegener's granulomatosis.

Type 1 diabetes is a form of diabetes mellitus in which not enough insulin is produced, which results in high blood sugar levels in the body. The symptoms of type 1 diabetes include frequent urination, increased thirst, increased hunger, weight loss, blurry vision, feeling tired and poor healing. While the cause of type 1 diabetes is unknown, the underlying mechanism involves an autoimmune destruction of the insulin-producing beta cells in the pancreas.

Systemic lupus erythematosus, also known as lupus, is a disease in which the body's immune system mistakenly attacks healthy tissues in many parts of the body. Common symptoms include panful and swollen joints, fever, chest pain, hair loss, mouth ulcers, swollen lymph nodes, feeling tired, and a red rash most commonly on the face. While the cause of lupus is still unknown, it may involve both genetic and environmental factors. The mechanism of lupus involves an immune response by autoantibodies against a person's own tissues, which are most commonly anti-nuclear antibodies that result in inflammation.

Rheumatoid arthritis is a long-term autoimmune disease that primarily affects joints, typically resulting in warm, swollen and painful joints. Other symptoms include low red blood cell count, inflammation around the lungs and the heart, fever and low energy. While the cause of rheumatoid arthritis is not clear, it is believed to involve a combination of genetic and environment factors. The underlying mechanism involves the body's immune system mistakenly attacking the joints, resulting in inflammation and thickening of the joint capsule and also affecting the underlying bone and cartilage.

Multiple sclerosis is an autoimmune disease in which the insulating covers of nerve cells in the brain and spinal cord are damaged by a person's own immune system. The damage disrupts the ability of the nervous system to communicate, causing a range of symptoms including double vision, blindness in one eye, muscle weakness, trouble with sensation, or trouble with coordination. While the cause is not clear, the underlying mechanism of multiple sclerosis is thought to be destruction by the immune system. Proposed causes include genetic and environment factors.

While autoimmune diseases are pervasive, their cause is generally unclear. The human adaptive immune system, including both T cells and B cells, is capable of being reactive with self-antigens. But these self-reactive T cells and B cells are usually either killed prior to becoming active within the immune system, placed into a state of anergy, or removed from their role within the immune system by regulatory cells. When any one of these mechanisms fail, some self-reactive cells may become functional within the immune system and cause autoimmune diseases.

Transplant rejection occurs when grafted tissue is rejected by the recipient's immune system, which destroys the grafted tissue. The underlying mechanism of rejection involves a combination of an adaptive immune response via cellular immunity which is mediated by killer T cells and humoral immunity mediated by activated B cells. Some components of innate immune response, such as phagocytes and soluble immune protein, may also be involved.

Acute transplant rejection may be treated with immunosuppressive therapy. Immunosuppressive drugs include corticosteroids, such as prednisolone and hydrocortisone, calcineurin inhibitors and mTOR inhibitors.

C. Administration of the Fusion Protein

In some embodiments, the present disclosure provides methods of treating a disease in a subject, comprising administering to the subject a therapeutically effective amount of the fusion protein or pharmaceutical composition provided herein.

The therapeutically effective amount (when used alone or in combination with other agents such as chemotherapeutic agents) of a fusion protein provided herein will depend on various factors known in the art, such as for example type of disease to be treated, body weight, age, past medical history, present medications, state of health of the subject, immune condition and potential for cross-reaction, allergies, sensitivities and adverse side-effects, as well as the administration route and the type, the severity and development of the disease and the discretion of the attending physician or veterinarian. In certain embodiments, the fusion protein provided herein may be administered at a therapeutically effective dosage of about 0.001 mg/kg to about 100 mg/kg one or more times per day (e.g., about 0.001 mg/kg, about 0.3 mg/kg, about 0.5 mg/kg, about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg one or more times per day). In certain embodiments, the fusion protein is administered at a dosage of about 50 mg/kg or less, and in certain embodiments the dosage is 20 mg/kg or less, 10 mg/kg or less, 3 mg/kg or less, 1 mg/kg or less, 0.3 mg/kg or less, 0.1 mg/kg or less, or 0.01 mg/kg or less, or 0.001 mg/kg or less. In certain embodiments, the administration dosage may change over the course of treatment. For example, in certain embodiments the initial administration dosage may be higher than the subsequent administration dosages. In certain embodiments, the administration dosage may vary over the course of treatment depending on the reaction of the subject.

Dosage regimens may be adjusted to provide the optimum desired response (e.g., a therapeutic response). In certain embodiments, the fusion protein provided herein is administered to the subject at one time or over a series of treatments. In certain embodiments, the fusion protein provided herein is administered to the subject by one or more separate administrations, or by continuous infusion depending on the type and severity of the disease.

The fusion protein provided herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

In certain embodiments, the fusion proteins thereof provided herein may be administered in a controlled-release manner. A controlled-release parenteral preparations can be made as implants, oily injections or particulate systems (e.g. microspheres, microparticles, microcapsules, nanocapsules, nanospheres, and nanoparticles) (see Banga, A. J., Therapeutic Peptides and Proteins: Formulation, Processing, and Delivery Systems, Technomic Publishing Company, Inc., Lancaster, Pa., (1995); Kreuter, J., Colloidal Drug Delivery Systems, J. Kreuter, ed., Marcel Dekker, Inc., New York, N.Y., pp. 219-342 (1994); Tice & Tabibi, Treatise on Controlled Drug Delivery, A. Kydonieus, ed., Marcel Dekker, Inc. New York, N.Y., pp. 315-339, (1992)). In certain embodiments, the fusion protein disclosed herein may be administered in degradable or nondegradable polymeric matrices (see Langer, Accounts Chem. Res. 26:537-542, 1993).

In some embodiments, the fusion protein provided herein can be administered alone or in combination with one or more additional therapeutic agents or means. For example, the fusion protein provided herein may be administered in combination with a second therapy, such as radiation therapy, chemotherapy, targeted therapies, gene therapy, immunotherapy, hormonal therapy, angiogenesis inhibition, palliative care, surgery for the treatment of tumor (e.g., tumorectomy), one or more anti-emetics or other treatments for complications arising from chemotherapy, or a second therapeutic agent for use in the treatment of tumor or any medical disorder, for example, another antibody, therapeutic polynucleotide, chemotherapeutic agent(s), anti-angiogenic agent, cytokines, other cytotoxic agent(s), growth inhibitory agent(s). In certain of these embodiments, the fusion protein provided herein may be administered simultaneously with the one or more additional therapeutic agents, and in certain of these embodiments the fusion protein and the additional therapeutic agent(s) may be administered as part of the same pharmaceutical composition. However, a fusion protein administered “in combination” with another therapeutic agent does not have to be administered simultaneously with or in the same composition as the agent. A fusion protein administered prior to or after another agent is considered to be administered “in combination” with that agent as the phrase is used herein, even if the fusion protein and second agent are administered via different routes. Where possible, additional therapeutic agents administered in combination with the fusion proteins provided herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

This example shows the generation of antibody-IL-10 fusion proteins and the test of their functions in vitro.

A list of antibody-IL-10 fusion proteins were designed based on anti-human PD-1 7A4D (see U.S. Pat. No. 10,428,146 to Qui et al) and anti-human PD-L1 antibody 5G11 (see U.S. Pat. No. 10,435,470 to Zha et al), respectively. The antibody-IL-10 fusion proteins were expressed in CHO cells through transient transfections, and affinity purified. The IL-10 moiety was linked to the C-terminus of either the heavy or light chain of the corresponding antibody via a linker of (GGGGSGGGGS (SEQ ID NO:15)). As shown in Table 1, the expression yield of each antibody-IL-10 fusion protein was similar to that of the corresponding antibody.

Further, the binding kinetics and binding affinity of each antibody-IL-10 fusion protein to the corresponding target were measured by Biacore analysis. The Ka, Kd, and K_{D} values were similar between the antibody and antibody-IL-10 fusion protein regardless of the fusion format.

TABLE 1
Characterization of the binding kinetics of antibody-IL-10 fusion proteins
based on anti-human PD-1 antibody7A4D and anti-human PD-L1 antibody 5G11.

| | | | | Binding kinetics to target | | | |
|---|---|---|---|---|---|---|---|
| Name of | Target of | IL-10 fusion | Expression | Ka | Kd | K_{D} | |
| protein | antibody | format | titer (mg/L) | (1/Ms) | (1/s) | (nM) | chi² |
| 7A4D | PD-1 | N/A | 25 | 9.74E+05 | 8.06E−04 | 0.83 | 0.234 |
| 7A4D-HC- | PD-1 | Heavy chain | 14 | 8.78E+05 | 8.18E−04 | 0.93 | 0.442 |
| rhIL10 | | C-terminal | | | | | |
| 7A4D-LC- | PD-1 | Light chain | 55 | 9.44E+05 | 8.23E−04 | 0.87 | 0.301 |
| rhIL10 | | C-terminal | | | | | |
| 5G11 | PD-L1 | N/A | 23 | 2.65E+06 | 7.79E−04 | 0.29 | 0.407 |
| 5G11-HC- | PD-L1 | Heavy chain | 23 | 2.75E+06 | 7.60E−04 | 0.28 | 0.218 |
| rhIL10 | | C-terminal | | | | | |
| 5G11-LC- | PD-L1 | Light chain | 29 | 2.82E+06 | 7.75E−04 | 0.27 | 0.163 |
| rhIL10 | | C-terminal | | | | | |

For Biacore analysis, anti-human IgG Fc secondary antibody was diluted with sodium acetate buffer, pH 5.0 to 25 μg/mL and immobilized onto both reference and test channels on a CM5 chip using Amine Coupling Kit. Each antibody or antibody-IL-10 fusion protein was diluted with running buffer to 1-3 μg/mL and captured on different channels of the pre-immobilized CM5 chip for 60 s at a flow rate of 10 μl/min. Binding was measured by flowing the recombinant human PD-1 or PD-L1 antigen in HBS EP buffer at a concentration between 3.125 and 100 nM in 2-fold serially-dilution at a flow rate of 30 μl/min. The association time and dissociation time were set at 90 s and 400 s, respectively. The measured affinity data were fitted to 1:1 Langmuir binding model using Biacore T200 Evaluation software 3.1.

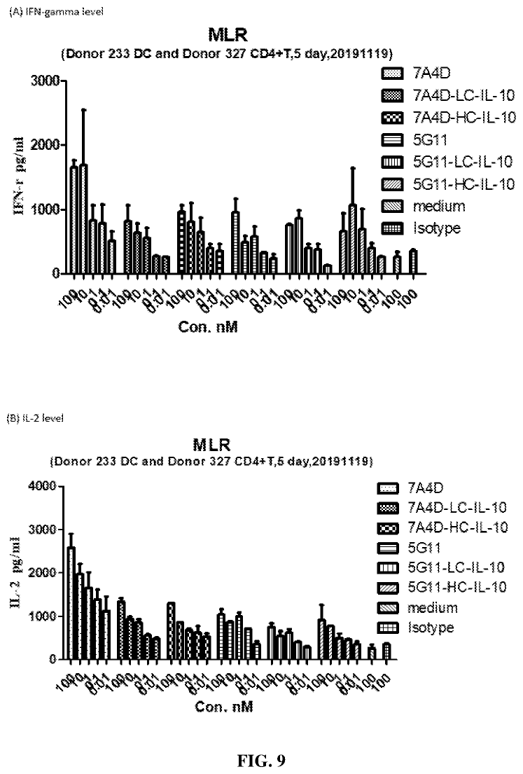

As shown in FIGS. 9A and 9B, both 7A4D and 5G11 antibody blocked the PD-L1-PD-1 pathway and induced IFN-gamma and IL-2 secretion of CD4⁺ effector T cells. The antibody-IL-10 fusion proteins based on either antibody elicited the same effect with the IL-10 moiety being linked to the Ig heavy or light chain C-terminus. Each antibody and the corresponding antibody-IL-10 fusion proteins showed the dose-dependent induction of cytokine production in a similar manner, suggesting that the linking of IL-10 to the antibody has minimal effect on the function of 7A4D and 5G11 antibody.

The inventor further tested whether the antibody-IL-10 fusion protein retains the IL-10 activity of inducing immune cell proliferation. IL-10 administration to MC/9 cells leads to cell proliferation in a dose-dependent manner. On a 96-well microtitre plate, 2.5×10⁴ MC/9 cells in 100 μl medium supplemented with recombinant Human IL-10 (rIL-10) or each antibody-IL-10 fusion protein to the designated final concentration were placed into each well. The maximum concentration of rIL-10 or fusion protein was 5,260 pmol/L. After 48 hours at 37° C., 5% CO₂, CellTiter 96® AQueous One Solution (Promega) was added to each assay well and the cell numbers were measured at absorbance at 490 nm. EC50 was calculated from the fitted curve of supplement-concentration versus OD 490 nm.

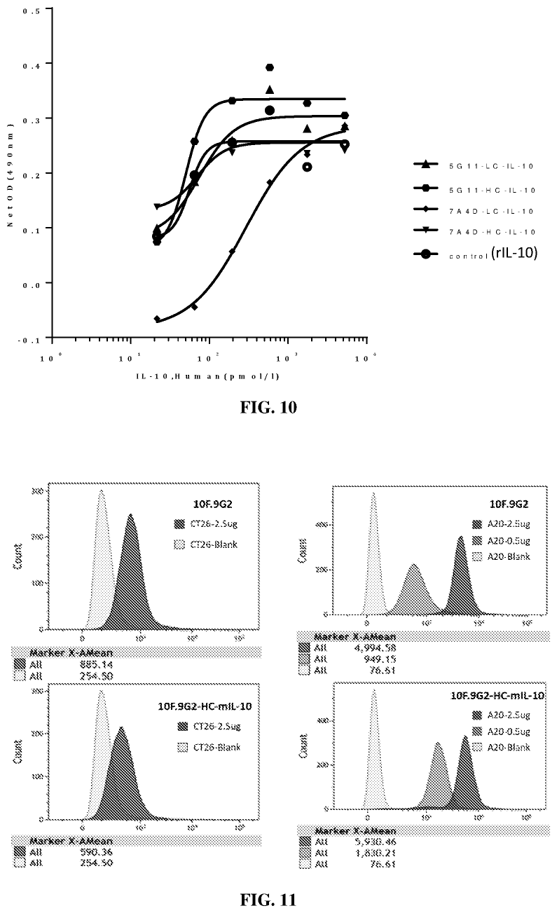

As shown in FIG. 10, IL-10 linked to the Ig heavy chain C-terminus of anti-PD-1 antibody 7A4D, but not light chain, induced MC/9 cell proliferation robustly, exhibiting a similar EC50 as human IL-10. Similarly, IL-10 linked to the C-terminus of heavy or light chain of the anti-PD-L1 antibody 5G11 induced MC/9 cell proliferation robustly, exhibiting a similar EC50 as human IL-10.

### Example 2

This example evaluates the in vivo therapeutic efficacy of antibody-IL-10 fusion proteins in syngeneic mouse tumor models. A fusion protein based on a rat-anti-mouse PD-L1 monoclonal antibody 10F.9G2 was designed, expressed in CHO cells through transient transfection and affinity purified. The antibody 10F.9G2 is of IgG2a, κ-isotype, and the fusion protein contains a mouse IL-10 moiety fused at the C-terminus of Ig heavy chain. The expression yield of the antibody-IL-10 fusion protein in comparison with the 10F.9G2 antibody is listed in Table 2.

TABLE 2
Expression yield of anti-mouse-PD-L1
antibody-IL-10 fusion protein

| | Culture | Final | Expression |
|---|---|---|---|
| | volume | yield | titer |
| Protein name | (ml) | (mg) | (mg/L) |
| 10F.9G2 (un-conjugated) | 200 | 27.265 | 136 |
| 10F.9G2-HC-mIL-10 | 600 | 32.88 | 55 |

Mouse tumor cell line A20 and CT26 express PD-L1 on the surface at a very high and low level, respectively. Anti-PD-L1 (10F.9G2) antibody and anti-PD-L1(10F.9G2) antibody-HC-mIL-10 fusion protein were each labelled with PE using a R-Phycoerythrin Conjugation Kit (Abcam). Prior to staining, A20 and CT26 cells were treated with 1 μg/mL Mouse BD Fc Block (BD Biosciences) at 4° C. for 5 min. Various amount of PE-labelled proteins as specified was used to stain treated cells in a total volume of 100 μl. Stained cells were analyzed on a LSRFortessa X-20 Flow Cytometer.

As shown in FIG. 11, anti-PD-L1(10F.9G2) antibody-HC-mIL-10 fusion protein showed dose-dependent staining of A20 cells, which has a high-expression of PD-L1, similar to the anti-PD-L1 (10F.9G2) antibody. For CT26 cells which expresses PD-L1 poorly, both anti-PD-L1 (10F.9G2) antibody and anti-PD-L1(10F.9G2) antibody-HC-mIL-10 fusion protein showed modest cell staining when a large amount of labelled protein was used (2.5 μg). These data suggest the fusion of IL-10 has little effect on the binding of anti-mouse PD-L1 antibody to its target.

A number of syngeneic mouse tumor models can be used to evaluate the therapeutic efficacy of anti-PD-L1(10F.9G2) antibody-HC-mIL-10 fusion protein in comparison with un-conjugated anti-PD-L1(10F.9G2) antibody and vehicle control. Such models include those established using CT26, A20, Hepa1-6, MBT-2, B16-F10 tumor cell lines.

For CT26 syngeneic tumor model, 5×10⁵ CT26 tumor cells were injected subcutaneously at the right rear flank region of female BALB/c mouse. Tumors were allowed to reach a size of 100-250 mm³ before treatment was started. For each treatment group, 8 mice were used and the variation of tumor volume in individual mouse across different groups was kept very similar. For treatment, anti-PD-L1(10F.9G2) antibody (6 mg/kg, 10 ul/g), anti-PD-L1(10F.9G2) antibody-HC-mIL-10 fusion protein (7 mg/kg, 10 ul/g), or vehicle only (PBS buffer, 10 ul/g) control was administered intraperitoneally at a site distant from the tumor with a frequency of twice per week for two weeks. Tumor growth was monitored 2-3 times per week using electronic calipers following each administration. Additionally, tumor tissues were harvested at designated experimental endpoints for performing immunohistochemistry on several inflammatory cell markers including CD45, CD4, CD8, and F4/80.

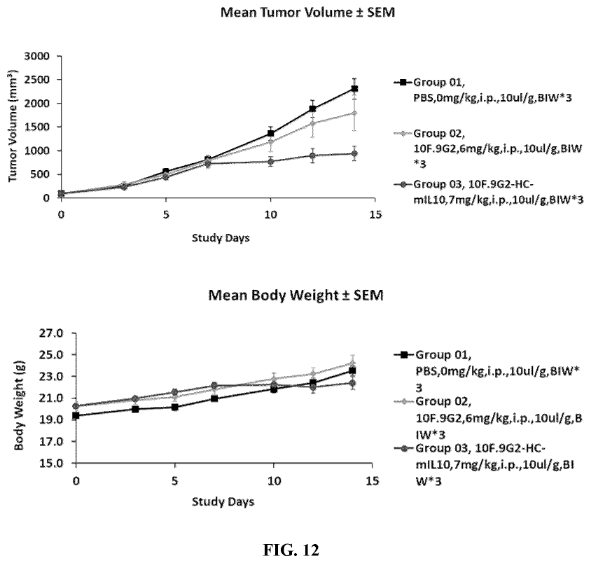

As shown in FIG. 12, following systemic treatment, anti-PD-L1(10F.9G2) antibody-HC-mIL-10 fusion protein led to more pronounced tumor growth inhibition than anti-PD-L1(10F.9G2) antibody which only showed modest anti-tumor effect, whereas continuous tumor growth was observed in the vehicle control group. Further, no obvious body weight loss was observed in all mice in treatment groups, suggesting little systemic toxicity elicited by using antibody alone or antibody-IL-10 fusion protein.

1. A protein comprising:
(a) an antibody or antigen binding fragment specifically binds to an immune checkpoint protein, said antibody or antigen-binding fragment comprising
(1) a heavy chain variable domain, and
(2) a light chain variable domain; and
(b) an interleukin-10 (IL-10) molecule operably linked to the antibody or antigen-binding fragment.

2. The protein of claim 1, wherein the immune checkpoint protein is PD-1 or PD-L1.

3. The protein of claim 1, wherein the antibody comprises a Fc region.

4. The protein of claim 1, wherein the antigen-binding fragment is an F(ab′)2 fragment or a single chain variable fragment (scFv).

5. The protein of claim 1, wherein the antibody or antigen-binding fragment comprises (1) a heavy chain comprising the heavy chain variable domain and (2) a light chain comprising the light chain variable domain.

6. The protein of claim 5, wherein the IL-10 molecule is linked to the heavy chain.

7. The protein of claim 6, wherein the IL-10 molecule is linked to C-terminal of the heavy chain.

8. The protein of claim 5, wherein the IL-10 molecule is linked to the light chain.

9. The protein of claim 8, wherein the IL-10 molecule is linked to C-terminal of the light chain.

10. The protein of claim 1, further comprising a linker that links the antibody or antigen-binding fragment thereof and the interleukin molecule.

11. The protein of claim 10, wherein the linker comprising the amino acid sequence (GGGGS)n (n=2-5).

12. An isolated polynucleotide encoding the protein of claim 1.

13. A vector capable of expressing a protein, comprising the isolated polynucleotide of claim 12.

14. A recombinant host cell suitable for producing a protein, comprising the vector of claim 13.

15. The recombinant host cell of claim 14, which is a mammalian cell line.

16. The recombinant host cell of claim 15, which is a CHO cell line.

17. A pharmaceutical composition comprising the protein of claim 1 and a pharmaceutically acceptable carrier.

18. A method for treating a disease in a subject in need thereof, comprising:
administering to the subject a therapeutically amount of the pharmaceutical composition of claim 17.

19. The method of claim 18, wherein the disease is a tumor, and the tumor is selected from the groups consisting of a lung cancer, a melanoma, a renal cancer, a liver cancer, a myeloma, a prostate cancer, a breast cancer, a colorectal cancer, a pancreatic cancer, a thyroid cancer, a hematological cancer, a leukemia and a non-Hodgkin's lymphoma.

20. (canceled)

21. The method of claim 18, wherein the disease is an immune related disorder, and the immune related disorder is selected from the group consisting of inflammatory bowel disease, Crohn's disease, ulcerative colitis, rheumatoid arthritis, psoriasis, type I diabetes, acute pancreatitis, uveitis, Sjogren's disease, Behcet's disease, sarcoidosis, graft versus host disease (GVHD), System Lupus Erythematosus, Vitiligo, chronic prophylactic acute graft versus host disease (pGvHD), HIV-induced vasculitis, Alopecia areata, Systemic sclerosis morphoea, and primary anti-phospholipid syndrome.

22. (canceled)


SEQUENCE LISTING (15 sequences)

SEQ ID NO: 1 - protein, 5 aa, Artificial Sequence
  Synthetic
NFGMN

SEQ ID NO: 2 - protein, 17 aa, Artificial Sequence
  Synthetic
WISGYTREPTYAADFKG

SEQ ID NO: 3 - protein, 5 aa, Artificial Sequence
  Synthetic
DVFDY

SEQ ID NO: 4 - protein, 15 aa, Artificial Sequence
  Synthetic
RASESVDNYGYSFMN

SEQ ID NO: 5 - protein, 7 aa, Artificial Sequence
  Synthetic
RASNLES

SEQ ID NO: 6 - protein, 8 aa, Artificial Sequence
  Synthetic
QQSNADPT

SEQ ID NO: 7 - protein, 5 aa, Artificial Sequence
  Synthetic
TYGVH

SEQ ID NO: 8 - protein, 16 aa, Artificial Sequence
  Synthetic
VIWRGVTTDYNAAFMS

SEQ ID NO: 9 - protein, 8 aa, Artificial Sequence
  Synthetic
LGFYAMDY

SEQ ID NO: 10 - protein, 11 aa, Artificial Sequence
  Synthetic
KASQSVSNDVA

SEQ ID NO: 11 - protein, 7 aa, Artificial Sequence
  Synthetic
YAANRYT

SEQ ID NO: 12 - protein, 9 aa, Artificial Sequence
  Synthetic
QQDYTSPYT

SEQ ID NO: 13 - protein, 160 aa, Mus musculus
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 14 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 15 - protein, 10 aa, Artificial Sequence
  Synthetic
GGGGSGGGGS